(19) United States
(12) Patent Application Publication
(10) Pub. No.: US 2017/0364633 A1
(43) Pub. Date: Dec. 21, 2017

(54) METHODS AND SYSTEMS TO GENERATE NONCODING-CODING GENE CO-EXPRESSION NETWORKS

(71) Applicant: KONINKLIJKE PHILIPS N.V., EINDHOVEN (NL)

(72) Inventors: NILANJANA BANERJEE, ARMONK, NY (US); NEVENKA DIMITROVA, PELHAM MANOR, NY (US); SONIA CHOTHANI, EINDHOVEN (NL); WILHELMUS FRANCISCUS JOHANNES VERHAEGH, EINDHOVEN (NL); YEE HIM CHEUNG, NEW YORK, NY (US)

(21) Appl. No.: 15/533,407

(22) PCT Filed: Dec. 7, 2015
(86) PCT No.: PCT/IB2015/059389

§ 371 Date: Jun. 6, 2017

Related U.S. Application Data
(60) Provisional application No. 62/090,127, filed on Dec. 10, 2014.

(51) Int. Cl. G06F 19/20; G06F 19/12; C12Q 1/68; G06F 19/18

(52) U.S. Cl. CPC G06F 19/20; G06F 19/18; G06F 19/12; C12Q 1/6883; C12Q 2600/112; C12Q 2600/118; C12Q 2600/178; C12Q 2600/158

(57) ABSTRACT

A method of identifying co-expressed coding and noncoding genes is disclosed. The method may include receiving genetic sequences, mapping the genetic sequences to known coding and noncoding genes, correlating the mapped genes, and generating a co-expression network. A system for generating a co-expression network and providing the co-expression network to a user on a display is disclosed. The system may include a memory, one or more processors, one or more databases, and a display.

### BACKGROUND

Long noncoding RNAs (lncRNAs) belong to a recently discovered class of transcripts that is suspected to have a wide range of roles in cellular functions including epigenetic silencing, transcriptional regulation, RNA processing and RNA modification. However, the precise transcriptional mechanisms and the interactions with coding RNAs (genes) are not well understood because they have not been annotated and are difficult to measure.

While most of the transcribed genome codes for proteins, a sizable proportion of the genome generates RNA transcripts do not code for proteins. A special class of noncoding RNA, long noncoding RNA (lncRNA) (>200 nucleotides long) has been shown to influence a wide variety of cellular functions including epigenetic silencing, transcriptional regulation, RNA processing and RNA modification. However, the precise transcriptional mechanisms of lncRNAs and their interactions with coding RNA are not well understood. Less than 1% of human lncRNAs (>8000) have been characterized. Regulation of protein-coding genes by overlapping, or nearby (cis) encoded, lncRNAs is central in cancer, cell cycle, and reprogramming. But activity where lncRNAs affect distant (trans) loci is also evident. To make matters more complicated, lncRNAs are expressed at low levels and are often specific to a particular tissue and condition. Better annotation of lncRNA expression patterns and the interplay with coding genes may improve the interpretation of genomic aberrations.

### SUMMARY

An exemplary method according to an embodiment of the disclosure may include receiving a plurality of RNA sequences in digital form in a memory, mapping at least one of the plurality of RNA sequences to a coding gene based on a set of coding genes in a database, mapping another at least one of the plurality of RNA sequences to a non-coding gene, correlating with at least one processor the coding gene and the non-coding gene, and generating a co-expression network based, at least in part, on results of the correlating.

Another exemplary method according to an embodiment of the disclosure may include receiving a plurality of RNA sequences in digital form in a memory, mapping some of the plurality of RNA sequences to coding genes based on a set of coding genes in a database, mapping another some of the plurality of RNA sequences to non-coding genes, determining variabilities of the coding genes and the non-coding genes, selecting the coding genes and non-coding genes that have variabilties above a threshold value, correlating with at least one processor the selected coding genes and the non-coding genes, and generating a co-expression network based, at least in part, on results of the correlating.

An exemplary system according to an embodiment of the disclosure may include at least one processor, a memory accessible to the at least one processor, the memory may be configured to store genetic sequences in digital form, a database accessible to the at least one processor, a display coupled to the at least one processor, and a non-transitory computer readable medium encoded with instructions that, when executed, may cause the at least one processor to: receive the genetic sequences from the memory, map some of the genetic sequences to coding genes based on a set of coding genes in a database, map another some of the genetic sequences to non-coding genes, calculate variabilities of the coding genes and the non-coding genes, select the coding genes and non-coding genes that have variabilties above a threshold value, correlate with at least one processor the selected coding genes and the non-coding genes to determine a co-expression of the selected coding genes and non-coding genes, generate a co-expression network based, at least in part, on the co-expression, and provide the co-expression network to a user on the display.

### BRIEF DESCRIPTION OF THE DRAWINGS

FIG. 1 is a functional block diagram of a system according to an embodiment of the disclosure.

FIG. 2 is an example gene co-expression network according to an embodiment of the disclosure.

FIG. 3 is a flow chart of a method according to an embodiment of the disclosure.

### DETAILED DESCRIPTION

The following description of certain exemplary embodiments is merely exemplary in nature and is in no way intended to limit the invention or its applications or uses. In the following detailed description of embodiments of the present systems and methods, reference is made to the accompanying drawings which form a part hereof, and in which are shown by way of illustration specific embodiments in which the described systems and methods may be practiced. These embodiments are described in sufficient detail to enable those skilled in the art to practice the presently disclosed systems and methods, and it is to be understood that other embodiments may be utilized and that structural and logical changes may be made without departing from the spirit and scope of the present system.

The following detailed description is therefore not to be taken in a limiting sense, and the scope of the present system is defined only by the appended claims. The leading digit(s) of the reference numbers in the figures herein typically correspond to the figure number, with the exception that identical components which appear in multiple figures are identified by the same reference numbers. Moreover, for the purpose of clarity, detailed descriptions of certain features will not be discussed when they would be apparent to those with skill in the art so as not to obscure the description of the present system.

Comparing transcript signals for RNA that encodes for genes, referred to herein as coding RNA and noncoding RNA (e.g., lncRNA) presents a problem for bioinformatics research. The distributions of coding RNA (coding genes) and noncoding RNA (noncoding genes) expression may differ for the low range and the high range values. The expression disparity may be due to a biological process and/or due to an experimental bias. To infer gene-noncoding gene interactions an appropriate similarity measure should allow for differences in scale of expression distribution.

While some noncoding genes have been characterized carefully for their role in cancer, systematic and principled approaches to map interactions of coding and noncoding genes are limited. Since noncoding RNAs were not well-known and unannotated, noncoding RNAs were not incorporated in previous high throughput measuring technologies (e.g., microarray).

RNA sequencing (RNAseq) has emerged as a powerful approach to profile a transcriptome without prior knowledge of the transcriptome. It may allow discovery and monitoring of additional coding and noncoding genes. As a result, with RNAseq data, it may be possible to detect many previously unknown noncoding genes. Since noncoding genes have lower levels of expression and higher variability, care should be taken as to how to integrate the two groups of RNA sequences, coding RNA and noncoding RNA, as erroneous methodologies may lead to inaccurate determination of interactions. These false interactions may lead to poor clinical decision making.

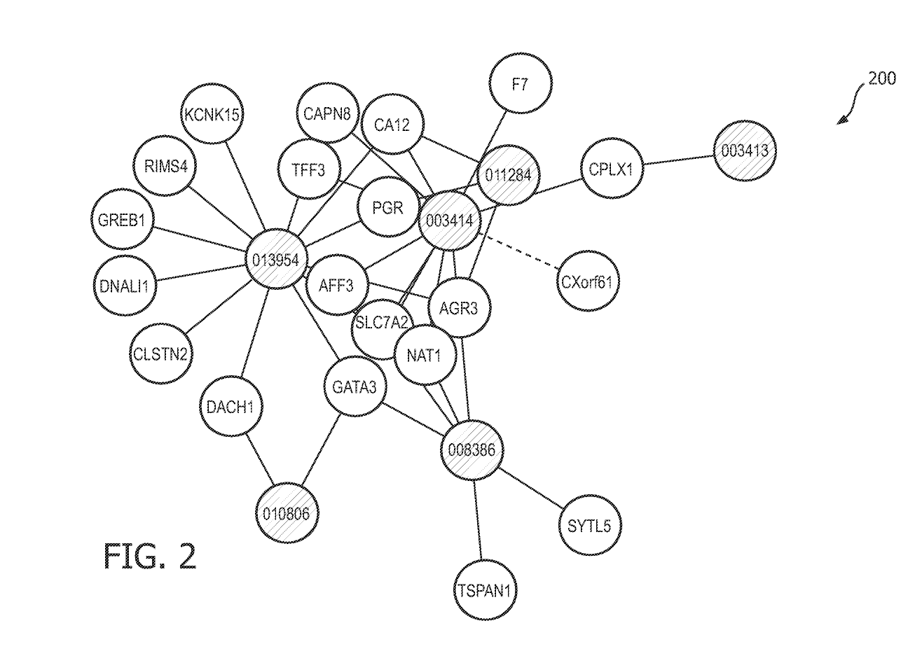

Given the observed discrepancy in expression level distribution among the coding and noncoding genes, an appropriate similarity measure may be used to properly associate a coding gene and a noncoding gene. Appropriately associated coding gene-noncoding gene pairs may be used to generate a co-expression network. A co-expression network is a graph that provides a visual representation of correlations between the expressions of genes, proteins, and/or genetic sequences. FIG. 2, which will be described in greater detail below, is an example of a gene co-expression network. Each node represents a gene encoded by RNA or a noncoding gene RNA. Nodes for coding genes and noncoding genes that are found to be frequently expressed together (positive correlation) may be connected by a solid line. Coding genes and noncoding genes that are found to almost never be expressed together (negative correlation) may be connected by a dashed line. The lines connecting the nodes are typically referred to as edges. Coding genes and noncoding genes that do not show a pattern of co-expression may not be connected. A cluster of highly correlated coding genes and/or noncoding genes may be referred to as a module. Modules may be analyzed further for coding gene-noncoding gene interactions to determine gene regulatory pathways and/or novel targets for therapy.

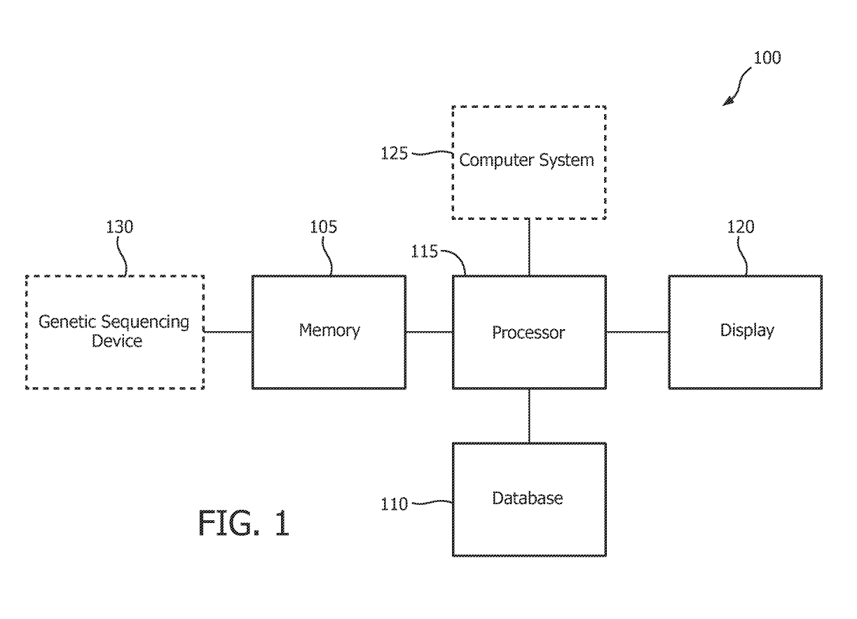

FIG. 1 is a functional block diagram of a system 100 according to an embodiment of the disclosure. The system 100 may be used to generate a co-expression network for coding genes and noncoding genes such as lncRNAs. A genetic sequence (e.g., RNA) in digital form may be included in memory 105. The genetic sequence may be received from a genetic sequencing machine in some embodiments. The genetic sequencing machine may have sequenced genetic material from a sample (e.g., blood, tissue). The memory 105 may be accessible to processor 115. The processor 115 may include one or more processors. The processor may be implemented as hardware, software, or combinations thereof. For example, in some embodiments, the processor may be an integrated circuit including circuits such as logic circuits and computational circuits. The circuits of the processor may operate to execute various operations and provide control signals to other circuits of a memory (such as memory 105. In some embodiments, the processor may be implemented as multiple processor circuits. The processor 115 may have access to a database 110 that includes one or more datasets (e.g., known genes, known noncoding genes, known lncRNAs). In some embodiments, the database 110 may include one or more databases. The processor 115 may provide the results of its calculations. In some embodiments, calculations may include mapping the genetic sequence to known noncoding genes and/or coding genes, calculating a correlation between the coding genes and noncoding genes, and/or generating a co-expression network. Other calculations may be performed by the processor 115. For example, the results (e.g., the generated co-expression network) may be provided to a display 120. The display 120 may be an electronic display that may be used to display the results to a user. The results may be provided to the database 110 for storing the results for later access.

In some embodiments, the system may also include other devices to provide the results, such as a printer. Optionally, processor 115 may further access a computer system 125. The computer system 125 may include additional databases, memories, and/or processors. The computer system 125 may be a part of system 100 or remotely accessed by system 100. In some embodiments, the system 100 may also include a genetic sequencing device 130. The genetic sequencing device 130 may process a biological sample (e.g., genetic isolate of a tumor biopsy, cheek swab) to generate a genetic sequence and produce the digital form of the genetic sequence to provide to memory 105.

The processor 115 may be configured to map received genetic sequences to known coding and noncoding genes, which may be stored in the database 110 in some embodiments. The processor 115 may be configured to correlate coding genes and noncoding genes to generate a co-expression network. The processor 115 may be configured to provide the co-expression network to the display 120, the database 110, memory 105, and/or computer system 125. In some embodiments, the processor 115 may be configured to calculate variabilities of expression of the coding genes and noncoding genes. The variability may be the variance in expression level across one or more samples from which the genetic sequences were obtained. The coding genes and noncoding genes having variabilities above a threshold value may be selected for inclusion in the co-expression network. In some embodiments, when the processor 115 includes more than one processor, the processors may be configured to perform different calculations to determine the co-expression network and/or perform calculations in parallel. In some embodiments, a non-transitory computer readable medium may be encoded with instructions that, when executed, cause the processor 115 to perform one or more of the above functions.

In some embodiments, the processor 115 may be configured to calculate more than one co-expression network. In some embodiments, one or more genetic sequences in the memory 105 may be added to the database 110. The genetic sequences may be added to one or more datasets in the database 110 and used to dynamically update the calculation of a co-expression network and/or used in subsequent calculations of a co-expression network.

The system 100 may allow for identification of key coding genes and noncoding genes and genomic aberrations in certain conditions and/or disease states (e.g., cancer, autoimmune diseases) by improving the accuracy of co-expression networks. This may lead to faster analysis of the most promising gene pathways for targets for novel therapies. Existing systems may provide a high percentage of false-positives for significance of co-expression of coding RNA and noncoding RNA, requiring extensive additional calculations, and/or time consuming review which reduces the ability to determine the most highly correlated co-expressed RNA. Determination of the co-expression network may allow the system 100, other systems, and/or users to make treatment and/or research decisions based on the co-expressed coding gene and/or noncoding gene pairs. The system 100 may select a druggable target (e.g., protein receptor, mRNA) and/or disease treatment based on the co-expression network by identifying a gene pathway that may be disrupted by a drug. For example, certain angiogenic gene pathways may be disrupted by rapamycin which may reduce blood vessel growth in tumors. The system 100 may be used to stratify patients based on the co-expression network. For example, patients whose tissue samples show a particular gene co-expression pattern may be identified as having conditions that are more or less severe, susceptible to treatment, and/or suitable for a clinical trial. The system 100 may be used in a research lab, a hospital, and/or other environment. A user may be a disease researcher, a doctor, and/or other clinician.

Once genetic sequences from samples (e.g., tissue biopsies, blood, cultured cells) are received, they may be mapped to known coding genes and noncoding genes. Known coding genes and noncoding genes may be stored in one or more databases. Optionally, the mapped genes may be analyzed for variability in expression. That is, genes that have a variance in rates of expression across samples. Coding genes and noncoding genes that have high variability in expression may be more likely to depend on the expression and/or suppression of other coding genes and/or noncoding genes. Conversely, coding genes and noncoding genes with uniform expression across samples may be more likely to be independent of other gene expression. For example, if a gene is expressed higher in benign tissue than in tumor tissue, the suppression of that gene's expression in tumors may play a role in tumor progression. A cancer researcher may be interested in finding what other coding genes or noncoding genes may be linked to its suppression. Continuing the example, a gene expressed equally in benign tissue samples and tumor tissue samples may not be likely to play a role in tumor development. In some embodiments, only mapped coding genes and noncoding genes having a variability above a threshold value (e.g., 75^{th} percentile, 90^{th} percentile) may be selected for further analysis. Variance in gene expression may be calculated using known statistical techniques.

After mapping, the coding genes and noncoding genes are exhaustively paired (i.e., all coding genes and noncoding genes are paired with all other coding genes and noncoding genes) and their similarities are analyzed. An appropriate similarity measure for the data should be used. An incorrect similarity measure relative to the data may lead to the derivation of erroneous interactions. Correlation analysis may provide an accurate similarity value for coding gene-noncoding gene pairs where expression of the coding gene is much higher than the noncoding gene. Correlation analysis may also be insensitive to whether the genes are cis (nearby) or trans (distant) to one another in the genome. An example of a correlation similarity measure that may be used for analysis is the Pearson correlation:

$$PCC ( g , l ) = \frac{Cov ( g , l )}{{σ}_{g} {σ}_{i}} \qquad Equation ( 1 )$$

where σ is the standard deviation and Cov is the covariance. The calculated correlation values for all of the coding gene and noncoding gene pairs may then be used to generate a co-expression network.

Each genetic sequence used to generate the exhaustive coding-coding, coding-noncoding, and noncoding-noncoding gene pairs are analyzed by the similarity measure and the properties of these three groups are characterized by comparing the distribution of the correlation-based similarity measure. Based on the distribution of values for the correlations, thresholds may be selected for generating a co-expression network. For example, only pairs with a correlation above the 99^{th} percentile may be selected for inclusion in the gene co-expression network. In another example, a correlation value over 0.7 may be selected for determining pairs included in the gene co-expression network. The pairs and the associated correlation values may be provided to a co-expression network software program. The co-expression network software program may construct and provide a graphical representation of the co-expression network on a display based on the received pairs and associated correlation values. An example of a co-expression network software package that may be used is Cytoscape.

FIG. 2 is an example co-expression network 200 according to an embodiment of the disclosure. The co-expression network 200 includes noncoding genes identified from lncRNAs and coding genes from RNAs received from breast tumor biopsies. The nodes having numbers starting with zero (‘0’) as labels represent lncRNAs (noncoding genes) and the nodes having labels starting with a letter represent coding genes. The edges connecting the nodes may be based on the calculated correlation values. In some embodiments, the length of the edge may be inversely proportional to how closely two nodes are correlated. A module may be two or more nodes connected by short edges in some embodiments. For example, nodes PGR, 003414, and 011284 may be considered a module in some embodiments. Optionally, groups of highly correlated nodes, modules, may be identified by a Markov clustering algorithm or other known clustering algorithm. In the example shown in FIG. 2, the co-expression network 200 may be used to start identifying putative lncRNA partners of known gene players in breast cancer as candidates for experimental validation. For example, TFF3 and ARG3 genes are involved in differentiation in estrogen receptor positive breast tumors are linked by edges to lncRNA 013954 and lncRNA 008386 respectively. The co-expression network 200 shows that the expression of TFF3 and 013954 may be correlated, and the expression of ARG3 and 008386 may be correlated. The lncRNAs connected to the genes may play a role in the regulating the expression of the TFF3 and ARG3 genes.

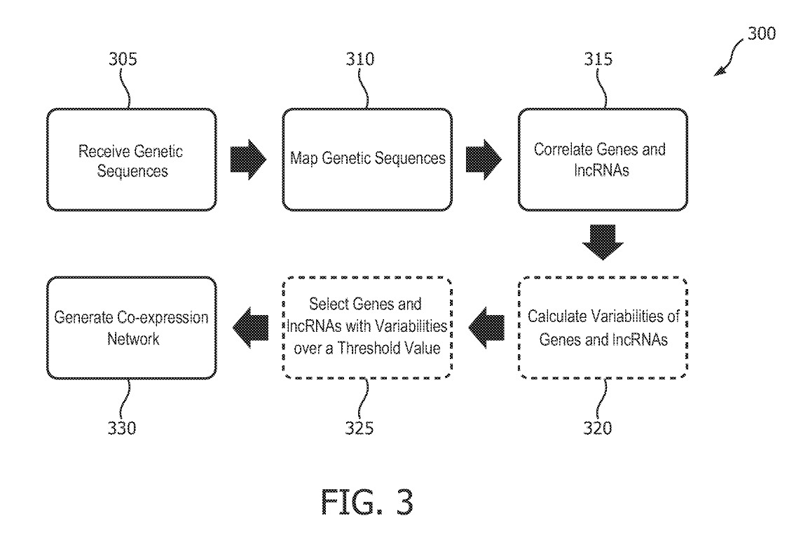

FIG. 3 is a flow chart of a method 300 according to an embodiment of the disclosure. In an embodiment of the invention, the method 300 may be implemented by the system 100 previously described with reference to FIG. 1. The method 300 may be used to generate a co-expression network for coding and noncoding genes. Genetic sequences may be received at Block 305. In some embodiments, the genetic sequences may be in digital form that may be stored in a computer-readable form. The genetic sequences may be stored in a volatile and/or nonvolatile memory. For example, the genetic sequence may be stored in digital form in memory 105 of system 100. The genetic sequences may be received from a genetic sequencing machine. In some embodiments, the genetic sequences may be RNA sequences.

At Block 310, the genetic sequences may be mapped to known coding genes and noncoding genes. In some embodiments, the noncoding genes may be long noncoding RNAs (lncRNAs). The known coding genes and noncoding genes may be stored in one or more databases. For example, coding genes and noncoding genes may be stored in database 110 of system 100. The genetic sequences may be mapped by one or more processors that have access to the memory and the database. The mapped coding and noncoding genes may be correlated to one another at Block 315. Correlations may be calculated for an exhaustive set of pairs for all the coding and noncoding genes. The correlations may be calculated by one or more processors in some embodiments. The mapping an correlation calculations may be performed by a processor, for example, processor 115 of system 100.

At Block 330, a co-expression network of the coding and noncoding genes may be generated by one or more processors. The co-expression network may be based on the correlation values calculated for the exhaustive set of pairs. In some embodiments, only pairs having a correlation value above a threshold value may be included in the co-expression network. In some embodiments, the co-expression network may be provided to a display accessible to the one or more processors. The co-expression network may be displayed on the display for viewing. For example, display 120 of system 100.

Optionally, in some embodiments of the inventions, one or both of the steps of Blocks 320 and 325 may be included in the method 300. The variability of expression of mapped coding and noncoding genes may be calculated as shown in Block 320. The variability may be the variance in expression level across one or more samples from which the genetic sequences were obtained. At Block 325, the mapped coding and noncoding genes having a variability above a threshold value may be selected for inclusion in the co-expression network. In some embodiments, Blocks 320 and 325 may be performed prior to Block 315. The variability may be calculated by one or more processors in some embodiments. For example, a processor such as processor 115 of system 100 may be used.

Of course, it is to be appreciated that any one of the above embodiments or processes may be combined with one or more other embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims

What is claimed is:

1. A method of identifying co-expressed coding and noncoding genes, the method comprising:
receiving a plurality of RNA sequences in digital form in a memory;
mapping at least one of the plurality of RNA sequences to a coding gene based on a set of coding genes in a database;
mapping another at least one of the plurality of RNA sequences to a non-coding gene;
correlating with at least one processor the coding gene and the non-coding gene; and
generating a co-expression network based, at least in part, on results of the correlating.

2. The method of claim 1, wherein correlating the coding gene and non-coding gene comprises applying a Pearson correlation.

3. The method of claim 1, further comprising generating a module based at least in part, on the co-expression network.

4. The method of claim 1, wherein generating the module includes applying a Markov cluster algorithm.

5. The method of claim 1, further comprising identifying a coding gene and non-coding gene partner based, at least in part, on the co-expression network.

6. The method of claim 5, wherein the coding gene and non-coding gene partner is in a gene expression pathway.

7. The method of claim 5, wherein the coding gene and non-coding gene pair are cis.

8. The method of claim 5, wherein the coding gene and non-coding gene pair are trans.

9. The method of claim 1, further comprising determining a variability of the coding gene and a variability of the non-coding gene.

10. A method, comprising:
receiving a plurality of RNA sequences in digital form in a memory;
mapping some of the plurality of RNA sequences to coding genes based on a set of coding genes in a database;
mapping another some of the plurality of RNA sequences to non-coding genes;
determining variabilities of the coding genes and the non-coding genes;
selecting the coding genes and non-coding genes that have variabilties above a threshold value;
correlating with at least one processor the selected coding genes and the non-coding genes; and
generating a co-expression network based, at least in part, on results of the correlating.

11. The method of claim 10, wherein the threshold value is 75^{th} percentile.

12. The method of claim 10, further comprising correlating the selected coding genes to each other.

13. The method of claim 10, further comprising correlating the selected non-coding genes to each other.

14. The method of claim 10, wherein the mapping another some of the plurality of RNA sequences to non-coding genes is based on a set of non-coding genes in the database.

15. The method of claim 10, wherein the another some of the plurality of RNA sequences to non-coding genes comprise long non-coding RNA (lncRNA) sequences.

16. The method of claim 10, wherein the plurality of RNA sequences are from a disease state.

17. A system, comprising:
at least one processor;
a memory accessible to the at least one processor, the memory configured to store genetic sequences in digital form;
a database accessible to the at least one processor;
a display coupled to the at least one processor; and
a non-transitory computer readable medium encoded with instructions that, when executed, cause the at least one processor to:
receive the genetic sequences from the memory;
map some of the genetic sequences to coding genes based on a set of coding genes in a database;
map another some of the genetic sequences to non-coding genes;
calculate variabilities of the coding genes and the non-coding genes;
select the coding genes and non-coding genes that have variabilties above a threshold value;
correlate with at least one processor the selected coding genes and the non-coding genes to determine a co-expression of the selected coding genes and non-coding genes;
generate a co-expression network based, at least in part, on the co-expression; and
provide the co-expression network to a user on the display.

18. The system of claim 17, wherein the non-transitory computer readable medium encoded with instructions that, when executed, further cause the at least one processor to select a druggable target based, at least in part, on the co-expression network.

19. The system of claim 17, wherein the non-transitory computer readable medium encoded with instructions that, when executed, further cause the at least one processor to stratify patients based, at least in part, on the co-expression network.

20. The system of claim 17, wherein the non-transitory computer readable medium encoded with instructions that, when executed, further cause the at least one processor to select a disease treatment based, at least in part on the co-expression network.
